(19) United States
(12) Patent Application Publication
(10) Pub. No.: US 2026/0047809 A1
(43) Pub. Date: Feb. 19, 2026

(54) SYSTEMS AND METHODS FOR MEDICAL IMAGING

(71) Applicant: SHANGHAI UNITED IMAGING HEALTHCARE CO., LTD., Shanghai (CN)

(72) Inventors: Yuan BAO, Shanghai (CN); Wenjing CAO, Shanghai (CN)

(73) Assignee: SHANGHAI UNITED IMAGING HEALTHCARE CO., LTD., Shanghai (CN)

(21) Appl. No.: 19/368,002

(22) Filed: Oct. 24, 2025

Related U.S. Application Data
(63) Continuation of application No. PCT/CN2023/094324, filed on May 15, 2023.

(51) Int. Cl. A61B 6/03; A61B 6/00; A61B 6/40

(52) U.S. Cl. CPC A61B 6/032; A61B 6/4007; A61B 6/482; A61B 6/5205; A61B 6/5235

(57) ABSTRACT

The present disclosure provides imaging systems and methods. The imaging systems may include a first imaging source, a second imaging source, and at least one detector. The first imaging source and the second imaging source may be configured to irradiate a subject simultaneously. The first imaging source may be configured to emit first radiation rays having a plurality of first energy levels for irradiating a subject in a first scanning angle range, the second imaging source may be configured to emit second radiation rays having a plurality of second energy levels for irradiating the subject in a second scanning angle range, and the second scanning angle range may be different from the first scanning angle range. The at least one detector may be configured to collect scan data of the subject by detecting the first radiation rays and the second radiation rays after passing through the subject.

### CROSS-REFERENCE TO RELATED APPLICATIONS

This application is a Continuation of International Application No. PCT/CN2023/094324, filed on May 15, 2023, the entire contents of which are incorporated herein by reference.

### TECHNICAL FIELD

The present disclosure generally relates to the imaging field, and more particularly, relates to medical imaging techniques that combine multi-source imaging techniques and multi-energy imaging techniques.

### BACKGROUND

With the development of science and technology, computed tomography (CT) devices play an increasingly important role in the medical field, such as detection, diagnosis, and surgery. However, for some specific regions (e.g., regions with physiological motion, such as the heart), CT imaging has requirements for temporal resolution and accuracy (e.g., consistency of spatial structures between different CT images). Therefore, it is desirable to provide multi-source imaging devices and methods thereof, which may improve the temporal resolution and the accuracy.

### SUMMARY

In an aspect of the present disclosure, an imaging system is provided. The imaging system may include a first imaging source, a second imaging source, and at least one detector. The first imaging source and the second imaging source may be configured to irradiate a subject simultaneously. The first imaging source may be configured to emit first radiation rays having a plurality of first energy levels for irradiating a subject in a first scanning angle range, the second imaging source may be configured to emit second radiation rays having a plurality of second energy levels for irradiating the subject in a second scanning angle range, and the second scanning angle range may be different from the first scanning angle range. The at least one detector may be configured to collect scan data of the subject by detecting the first radiation rays and the second radiation rays after passing through the subject.

In some embodiments, the first imaging source and the second imaging source may be kV switching imaging sources.

In some embodiments, switching patterns of the first imaging source may be determined based on penetrating capacities of the plurality of first energy levels, and switching patterns of the second imaging source may be determined based on penetrating capacities of the plurality of second energy levels.

In some embodiments, the first imaging source and the second imaging source may be multi-beam imaging sources.

In some embodiments, the imaging system may further include a plurality of third imaging sources configured to irradiate the subject with the first imaging source and the second imaging source, simultaneously. Each of the plurality of third imaging sources may be configured to emit third radiation rays having one or more energy levels for irradiating the subject in a portion of a third scanning angle range, and the third scanning angle range may be different from the first scanning angle range and the second scanning angle range. The at least one detector may be further configured to collect the scan data of the subject by detecting the first radiation rays, the second radiation rays, and the third radiation rays after passing through the subject.

In some embodiments, the scan data of the subject may include a plurality of sets of scan data each of which corresponds to one of a plurality of energy levels. The plurality of energy levels may include the plurality of first energy levels and the plurality of second energy levels. The imaging system may further include a processing device configured to generate reconstruction images corresponding to the plurality of energy levels based on the plurality of sets of scan data.

In some embodiments, to generate reconstruction images corresponding to the plurality of energy levels based on the plurality of sets of scan data, the processing device may be configured to generate a plurality of sets of weighted scan data by weighting the plurality of sets of scan data using a weighting model, the weighting model indicating a corresponding relationship between weighting values and scanning angles; and generate the reconstruction images corresponding to the plurality of energy levels based on the plurality of sets of weighted scan data.

In some embodiments, the reconstruction images may further correspond to a target scanning angle range. To generate reconstruction images corresponding to the plurality of energy levels based on the plurality of sets of scan data, the processing device may be configured to generate a plurality of preliminary reconstruction images corresponding to the plurality of energy levels based on the plurality of sets of scan data; and generate the reconstruction images by processing the plurality of preliminary reconstruction images using at least one first image generation model corresponding to the target scanning angle range.

In some embodiments, the at least one first image reconstruction model may include a plurality of first image reconstruction models each of which corresponds to one of the plurality of energy levels. To generate the reconstruction images by processing the plurality of preliminary reconstruction images, the processing device may be configured to: for each of the plurality of energy levels, select the first image reconstruction model of the energy level from the plurality of first image reconstruction models; and generate the reconstruction image corresponding to the energy level by processing the preliminary reconstruction image of the energy level using the selected first image reconstruction model.

In some embodiments, to generate reconstruction images corresponding to the plurality of energy levels based on the plurality of sets of scan data, the processing device may be configured to: for each of the plurality of energy levels, determine whether an actual energy level of a corresponding group of scan data satisfies a preset condition, the group of scan data including a portion of the plurality of sets of scan data corresponding to the energy level; in response to determining that the energy level of the corresponding group of scan data satisfies the preset condition, generate the reconstruction image corresponding to the energy level based on the group of scan data of the energy level; or in response to that the energy level of the corresponding group of scan data doesn't satisfy the preset condition, generate the reconstruction image corresponding to the energy level based on the group of scan data and a second image generation model corresponding to the energy level.

In some embodiments, the processing device may be further configured to obtain registered reconstruction images by performing a registration operation on the reconstruction images; and generate one or more material-specific images by performing a material separation on the registered reconstruction images.

In some embodiments, to obtain registered reconstruction images by performing a registration operation on the reconstruction images, the processing device may be further configured to determine a target energy level from the plurality of energy levels; for each energy level other than the target energy level, generate a processed image corresponding to the target energy level by processing the reconstruction image of the energy level using a third image generation model corresponding to the target energy level; and obtain the registered reconstruction images by performing the registration operation on the reconstruction image corresponding to the target energy level and the processed image of each energy level other than the target energy level.

In some embodiments, to process the reconstruction image of the energy level, the processing device may be further configured to process the reconstruction image of the energy level using a third image generation model corresponding to the target energy level.

In another aspect of the present disclosure, an imaging method is provided. The imaging method may be implemented on a computing device having at least one processor and at least one storage device. The method may include directing a first imaging source and a second imaging source to irradiate a subject simultaneously. The first imaging source may be configured to emit first radiation rays having a plurality of first energy levels for irradiating a subject in a first scanning angle range, the second imaging source may be configured to emit second radiation rays having a plurality of second energy levels for irradiating the subject in a second scanning angle range, and the second scanning angle range may be different from the first scanning angle range. The method may further include directing at least one detector to collect scan data of the subject by detecting the first radiation rays and the second radiation rays after passing through the subject.

In still another aspect of the present disclosure, a non-transitory computer readable medium is provided. The non-transitory computer readable medium may include executable instructions that, when executed by at least one processor, direct the at least one processor to perform an imaging method. The method may include directing a first imaging source and a second imaging source to irradiate a subject simultaneously. The first imaging source may be configured to emit first radiation rays having a plurality of first energy levels for irradiating a subject in a first scanning angle range, the second imaging source may be configured to emit second radiation rays having a plurality of second energy levels for irradiating the subject in a second scanning angle range, and the second scanning angle range may be different from the first scanning angle range. The method may further include directing at least one detector to collect scan data of the subject by detecting the first radiation rays and the second radiation rays after passing through the subject.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a schematic diagram illustrating an exemplary imaging system according to some embodiments of the present disclosure;

FIG. 2 is a block diagram illustrating exemplary hardware and/or software components of an exemplary computing device according to some embodiments of the present disclosure;

FIG. 3 is a schematic diagram illustrating exemplary hardware and/or software components of an exemplary mobile device on which a terminal may be implemented according to some embodiments of the present disclosure;

FIG. 4A is a schematic diagram illustrating an exemplary imaging device according to some embodiments of the present disclosure;

FIG. 4B is a schematic diagram illustrating another exemplary imaging device according to some embodiments of the present disclosure;

FIG. 4C is a schematic diagram illustrating another exemplary imaging device according to some embodiments of the present disclosure;

FIG. 5A is a schematic diagram illustrating an exemplary scanning process of an imaging device according to some embodiments of the present disclosure;

FIG. 5B is a schematic diagram illustrating an exemplary scanning process of an imaging device according to some embodiments of the present disclosure;

FIG. 6 is a block diagram illustrating an exemplary processing device according to some embodiments of the present disclosure;

FIG. 7 is a flowchart illustrating an exemplary process for generating one or more material-specific images according to some embodiments of the present disclosure;

FIG. 8A is a flowchart illustrating an exemplary process for generating reconstruction images of a subject according to some embodiments of the present disclosure;

FIG. 8B is a schematic diagram illustrating an exemplary process for generating reconstruction images of a subject according to some embodiments of the present disclosure;

FIG. 9 is a flowchart illustrating an exemplary process for generating reconstruction images corresponding to an energy level according to some embodiments of the present disclosure;

FIG. 10 is a flowchart illustrating an exemplary process for obtaining registered reconstruction images according to some embodiments of the present disclosure; and

FIG. 11 is a schematic diagram illustrating an exemplary weighting model according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that when a unit, engine, module, or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

In the present disclosure, the subject may include a biological object and/or a non-biological object. The biological object may be a human being, an animal, a plant, or a specific portion, organ, and/or tissue thereof. For example, the subject may include the head, the neck, the thorax, the heart, the stomach, a blood vessel, a soft tissue, a tumor, a nodule, or the like, or any combination thereof. In some embodiments, the subject may be a man-made composition of organic and/or inorganic matters that are with or without life. The terms “object” and “subject” are used interchangeably in the present disclosure.

In the present disclosure, the term “image” may refer to a two-dimensional (2D) image, a three-dimensional (3D) image, or a four-dimensional (4D) image (e.g., a time series of 3D images). In some embodiments, the term “image” may refer to an image of a region (e.g., a region of interest (ROI)) of a subject. In some embodiment, the image may be a medical image, an optical image, etc.

The present disclosure relates to systems and methods for medical imaging. The systems may include a first imaging source, a second imaging source, and at least one detector. The first imaging source and the second imaging source may be configured to irradiate a subject simultaneously, wherein the first imaging source is configured to emit first radiation rays having a plurality of first energy levels for irradiating a subject in a first scanning angle range, the second imaging source is configured to emit second radiation rays having a plurality of second energy levels for irradiating the subject in a second scanning angle range, and the second scanning angle range is different from the first scanning angle range. The plurality of first energy levels may be the same as the plurality of second energy levels. The at least one detector may be configured to collect scan data of the subject by detecting the first radiation rays and the second radiation rays after passing through the subject.

The systems and methods of the present disclosure may combine the multi-source imaging technique (by using a plurality of imaging sources) and the multi-energy imaging technique (by using radiation rays having a plurality of energy levels). Thus, the systems and methods of the present disclosure may have the advantages of both the multi-source imaging technique and the multi-energy imaging technique. The utilization of the multi-source imaging technique may improve the temporal resolution of the scan, and the utilization of the multi-energy imaging technique may provide additional information (accurate composition information) beyond images reconstructed based on scan data that is obtained with radiation rays having a single energy level. In some embodiments, the scan data may include sets of scan data of different energy levels. By setting the switching pattern of the imaging sources, the sets of scan data of different energy levels may be collected almost at the same time and have a relatively small spatial structure inconsistency. In this way, images reconstructed based on the sets of scan data may have a relatively high consistency. In some embodiments, one or more machine learning models may be adopted to improve the temporal resolution and/or data accuracy.

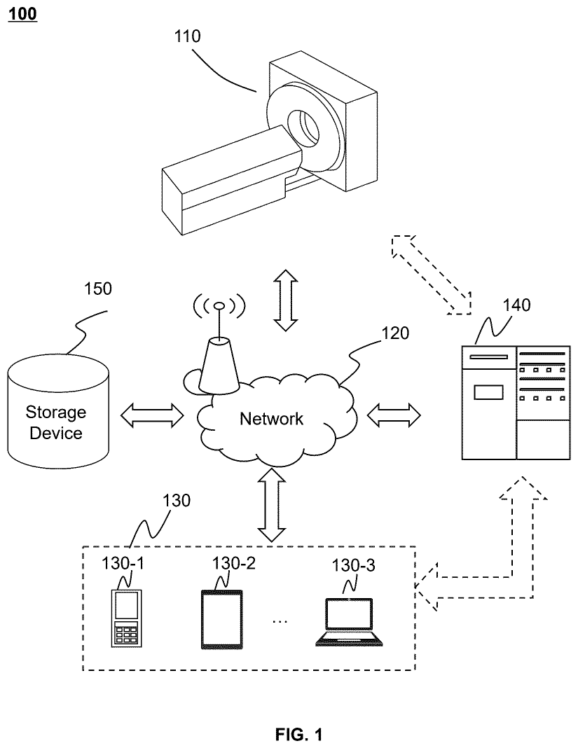

FIG. 1 is a schematic diagram illustrating an exemplary imaging system according to some embodiments of the present disclosure. As shown, the imaging system 100 may include an imaging device 110, a network 120, one or more terminals 130, a processing device 140, and a storage device 150. In some embodiments, the imaging device 110, the processing device 140, the storage device 150, and/or the terminal(s) 130 may be connected to and/or communicate with each other via a wireless connection (e.g., the network 120), a wired connection, or a combination thereof. The connection between the components in the imaging system 100 may be variable. Merely by way of example, the imaging device 110 may be connected to the processing device 140 through the network 120, as illustrated in FIG. 1. As another example, the imaging device 110 may be connected to the processing device 140 directly. As a further example, the storage device 150 may be connected to the processing device 140 through the network 120, as illustrated in FIG. 1, or connected to the processing device 140 directly.

The imaging device 110 may be configured to acquire scan data relating to at least one part of a subject. For example, the imaging device 110 may scan the subject or a portion thereof that is located within its detection region and generate scan data (e.g., an initial image) relating to the subject or the portion thereof. The scan data relating to at least one part of a subject may include an image (e.g., an image slice), projection data, or a combination thereof. In some embodiments, the imaging device 110 may include a single modality imaging device. For example, the imaging device 110 may include a computed tomography (CT) device. In some embodiments, the imaging device 110 may include a multi-modality imaging device. Exemplary multi-modality imaging devices may include a positron emission tomography-computed tomography (PET-CT) device, a CT-guided radiotherapy device, etc.

Merely by way of example, the imaging device 110 may be a CT device. The imaging device 110 may include a supporting assembly, a detector assembly, a detection region, a table, and a radiation emitting assembly. The supporting assembly may support the detector assembly and the radiation emitting assembly. The subject may be placed on the table and moved to the detection region along with a movement of the table. The radiation emitting assembly may emit a plurality of beams of radiation rays (e.g., X-rays) to the subject. In some embodiments, the radiation emitting assembly may include a plurality of imaging sources configured to irradiate the subject simultaneously. Each of the imaging sources may be responsible for a specific scanning angle range, and configured to emit radiation rays toward the subject in its corresponding scanning angle range. In some embodiments, each of the plurality of imaging sources may emit radiation rays having a plurality of energy levels so as to achieve muti-energy scanning.

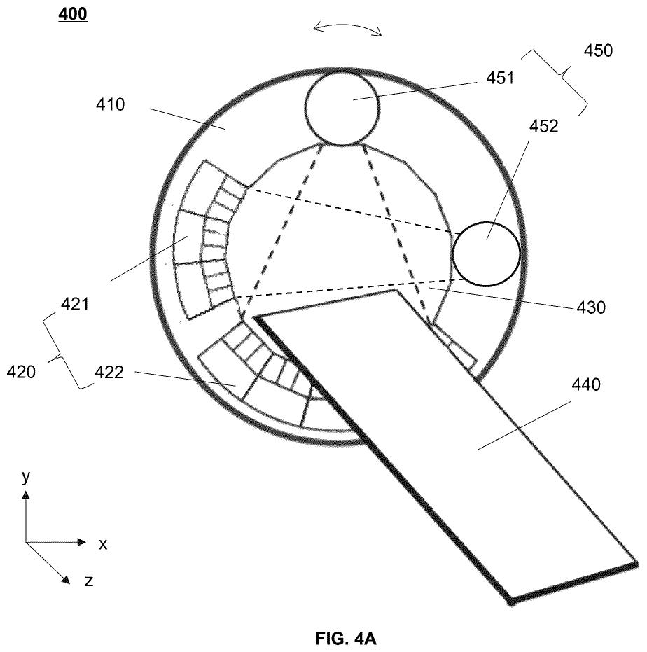

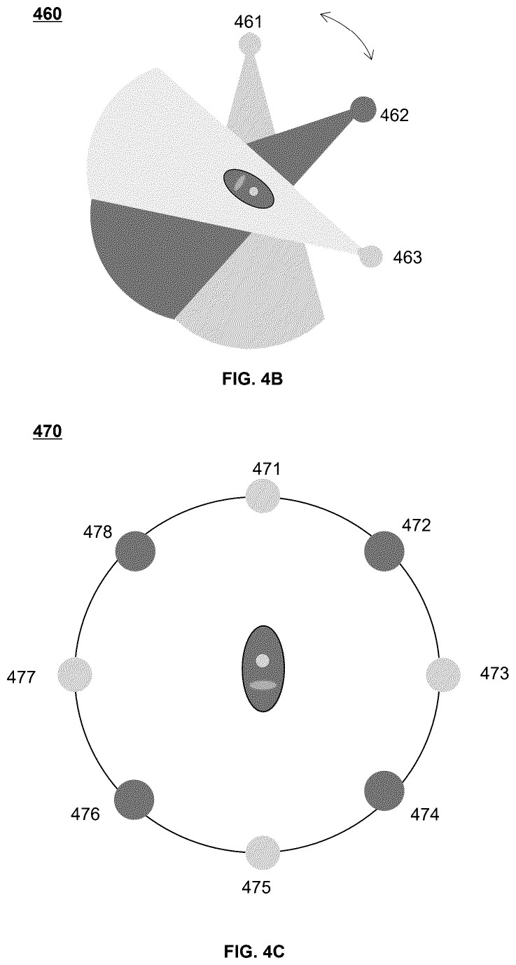

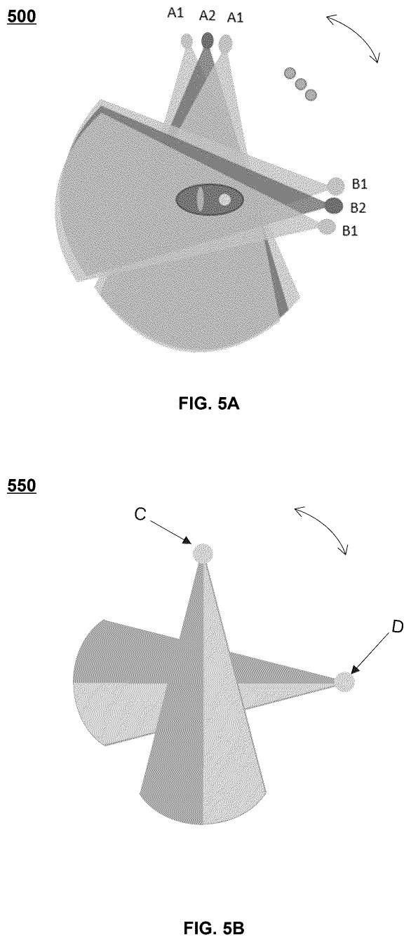

The detector assembly may collect scan data of the energy levels by detecting the radiation beam passing through the detection region. For example, the detector assembly may convert radiation photons of the radiation beam into an electronic signal. The electronic signal may be transmitted to a computing device (e.g., the processing device 140) for processing, or transmitted to a storage device (e.g., the storage device 150) for storage. In some embodiments, the detector assembly may include a plurality of detectors each of which corresponds to one of the plurality of imaging sources of the imaging device 110. More descriptions regarding an imaging device may be found elsewhere in the present disclosure (e.g., FIGS. 4A-4B and FIGS. 5A-5B, and the descriptions thereof).

The network 120 may include any suitable network that can facilitate the exchange of information and/or data for the imaging system 100. In some embodiments, one or more components (e.g., the imaging device 110, the terminal 130, the processing device 140, the storage device 150, etc.) of the imaging system 100 may communicate information and/or data with one or more other components of the imaging system 100 via the network 120. For example, the processing device 140 may obtain scan data from the imaging device 110 via the network 120. As another example, the processing device 140 may obtain user instructions from the terminal 130 via the network 120. In some embodiments, the network 120 may include one or more network access points. For example, the network 120 may include wired and/or wireless network access points such as base stations and/or internet exchange points through which one or more components of the imaging system 100 may be connected to the network 120 to exchange data and/or information.

The terminal(s) 130 may include a mobile device 130-1, a tablet computer 130-2, a laptop computer 130-3, or the like, or any combination thereof. In some embodiments, the mobile device 130-1 may include a smart home device, a wearable device, a mobile device, a virtual reality device, an augmented reality device, or the like, or any combination thereof. In some embodiments, the smart home device may include a smart lighting device, a control device of an intelligent electrical apparatus, a smart monitoring device, a smart television, a smart video camera, an interphone, or the like, or any combination thereof. In some embodiments, the wearable device may include a bracelet, a footgear, eyeglasses, a helmet, a watch, clothing, a backpack, a smart accessory, or the like, or any combination thereof. In some embodiments, the mobile device may include a mobile phone, a personal digital assistance (PDA), a gaming device, a navigation device, a point of sale (POS) device, a laptop, a tablet computer, a desktop, or the like, or any combination thereof. In some embodiments, the virtual reality device and/or the augmented reality device may include a virtual reality helmet, virtual reality glasses, a virtual reality patch, an augmented reality helmet, augmented reality glasses, an augmented reality patch, or the like, or any combination thereof. In some embodiments, the terminal(s) 130 may be part of the processing device 140.

The processing device 140 may process data and/or information obtained from one or more components (the imaging device 110, the terminal(s) 130, and/or the storage device 150) of the imaging system 100. For example, the processing device 140 may process scan data acquired by the imaging device 110 (e.g., scan data detected by the detector assembly of the imaging device 110) and generate a reconstruction image based on the scan data. In some embodiments, the processing device 140 may be a single server or a server group. The server group may be centralized or distributed. In some embodiments, the processing device 140 may be local or remote. For example, the processing device 140 may access information and/or data stored in the imaging device 110, the terminal(s) 130, and/or the storage device 150 via the network 120. As another example, the processing device 140 may be directly connected to the imaging device 110, the terminal(s) 130, and/or the storage device 150 to access stored information and/or data. In some embodiments, the processing device 140 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof.

In some embodiments, the processing device 140 may be implemented by a computing device. For example, the computing device may include a processor, a storage, an input/output (I/O), and a communication port. The processor may execute computer instructions (e.g., program codes) and perform functions of the processing device 140 in accordance with the techniques described herein. The computer instructions may include, for example, routines, programs, objects, components, data structures, procedures, modules, and functions, which perform particular functions described herein. In some embodiments, the processing device 140, or a portion of the processing device 140 may be implemented by a portion of the terminal 130.

The storage device 150 may store data/information obtained from the imaging device 110, the terminal(s) 130, and/or any other component of the imaging system 100. In some embodiments, the storage device 150 may include a mass storage, a removable storage, a volatile read-and-write memory, a read-only memory (ROM), or the like, or any combination thereof. For example, the mass storage may include a magnetic disk, an optical disk, a solid-state drive, etc. The removable storage may include a flash drive, a floppy disk, an optical disk, a memory card, a zip disk, a magnetic tape, etc. In some embodiments, the storage device 150 may store one or more programs and/or instructions to perform exemplary methods described in the present disclosure.

In some embodiments, the storage device 150 may be connected to the network 120 to communicate with one or more other components in the imaging system 100 (e.g., the processing device 140, the terminal(s) 130, etc.). One or more components in the imaging system 100 may access the data or instructions stored in the storage device 150 via the network 120. In some embodiments, the storage device 150 may be directly connected to or communicate with one or more other components in the imaging system 100 (e.g., the processing device 140, the terminal(s) 130, etc.). In some embodiments, the storage device 150 may be part of the processing device 140.

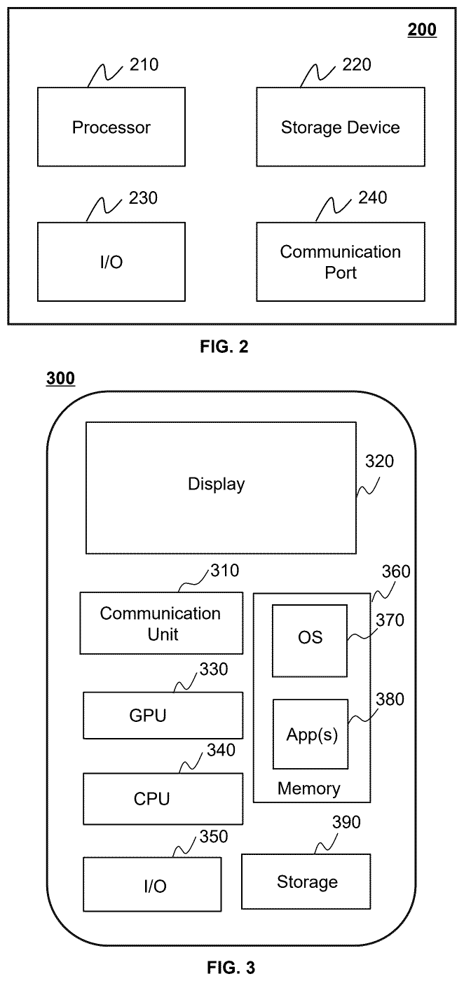

FIG. 2 is a schematic diagram illustrating exemplary hardware and/or software components of an exemplary computing device 200 according to some embodiments of the present disclosure. The computing device 200 may be configured to implement any component of the imaging system 100. For example, the imaging device 110, the terminal 130, the processing device 140, and/or the storage device 150 may be implemented on the computing device 200. Although only one such computing device is shown for convenience, the computer functions relating to the imaging system 100 as described herein may be implemented in a distributed fashion on a number of similar platforms, to distribute the processing load. As illustrated in FIG. 2, the computing device 200 may include a processor 210, a storage device 220, an input/output (I/O) 230, and a communication port 240.

The processor 210 may execute computer instructions (e.g., program codes) and perform functions of the processing device 140 in accordance with the techniques described herein. The computer instructions may include, for example, routines, programs, objects, components, signals, data structures, procedures, modules, and functions, which perform particular functions described herein. In some embodiments, the processor 210 may perform instructions obtained from the terminal 130 and/or the storage device 150. In some embodiments, the processor 210 may include one or more hardware processors, such as a microcontroller, a microprocessor, a reduced instruction set computer (RISC), an application-specific integrated circuits (ASICs), an application-specific instruction-set processor (ASIP), a central processing unit (CPU), a graphics processing unit (GPU), a physics processing unit (PPU), a microcontroller unit, a digital signal processor (DSP), a field-programmable gate array (FPGA), an advanced RISC machine (ARM), a programmable logic device (PLD), any circuit or processor capable of executing one or more functions, or the like, or any combinations thereof.

Merely for illustration, only one processor is described in the computing device 200. However, it should be noted that the computing device 200 in the present disclosure may also include multiple processors. Thus operations and/or method steps that are performed by one processor as described in the present disclosure may also be jointly or separately performed by the multiple processors. For example, if in the present disclosure the processor of the computing device 200 executes both operation A and operation B, it should be understood that operation A and operation B may also be performed by two or more different processors jointly or separately in the computing device 200 (e.g., a first processor executes operation A and a second processor executes operation B, or the first and second processors jointly execute operations A and B).

The storage device 220 may store data/information obtained from the imaging device 110, the terminal 130, the storage device 150, or any other component of the imaging system 100. In some embodiments, the storage device 220 may include a mass storage device, a removable storage device, a volatile read-and-write memory, a read-only memory (ROM), or the like, or any combination thereof. For example, the mass storage device may include a magnetic disk, an optical disk, a solid-state drive, a mobile storage device, etc. The removable storage device may include a flash drive, a floppy disk, an optical disk, a memory card, a ZIP disk, a magnetic tape, etc. The volatile read-and-write memory may include a random access memory (RAM). The RAM may include a dynamic RAM (DRAM), a double date rate synchronous dynamic RAM (DDR-SDRAM), a static RAM (SRAM), a thyristor RAM (T-RAM), and a zero-capacitor RAM (Z-RAM), etc. The ROM may include a mask ROM (MROM), a programmable ROM (PROM), an erasable programmable ROM (EPROM), an electrically erasable programmable ROM (EEPROM), a compact disk ROM (CD-ROM), a digital versatile disk ROM, etc. In some embodiments, the storage device 220 may store one or more programs and/or instructions to perform exemplary methods described in the present disclosure.

The I/O 230 may input or output signals, data, and/or information. In some embodiments, the I/O 230 may enable user interaction with the processing device 120. In some embodiments, the I/O 230 may include an input device and an output device. Exemplary input devices may include a keyboard, a mouse, a touch screen, a microphone, a camera capturing gestures, or the like, or a combination thereof. Exemplary output devices may include a display device, a loudspeaker, a printer, a projector, a 3D hologram, a light, a warning light, or the like, or a combination thereof. Exemplary display devices may include a liquid crystal display (LCD), a light-emitting diode (LED)-based display, a flat panel display, a curved screen, a television device, a cathode ray tube (CRT), or the like, or a combination thereof.

The communication port 240 may be connected to a network (e.g., the network 120) to facilitate data communications. The communication port 240 may establish connections between the processing device 140 and the imaging device 110, the terminal 130, the storage device 150, or any external devices (e.g., an external storage device, or an image/data processing workstation). The connection may be a wired connection, a wireless connection, or a combination of both that enables data transmission and reception. The wired connection may include an electrical cable, an optical cable, a telephone wire, or the like, or any combination thereof. In some embodiments, the communication port 240 may be a standardized communication port, such as RS232, RS485, etc. In some embodiments, the communication port 240 may be a specially designed communication port. For example, the communication port 240 may be designed in accordance with the digital imaging and communications in medicine (DICOM) protocol.

In some embodiments, the computing device 200 may further include a bus (not shown) configured to achieve the communication between the processor 210, the storage device 220, the I/O 230, and/or the communication port 240. The bus may include hardware, software, or both, which decouple the components of the computing device 200 to each other. The bus may include at least one of a data bus, an address bus, a control bus, an expansion bus, or a local bus. For example, the bus may include an accelerated graphics port (AGP) or other graphics bus, an extended industry standard architecture (EISA) bus, a front side bus (FSB), a hyper transport (HT) interconnection, an industry standard architecture (ISA) bus, a front side bus (FSB), an Infiniband interconnection, a low pin count (LPC) bus, a storage bus, a micro channel architecture (MCA) bus, a peripheral component interconnect (PCI) bus, a PCI-Express (PCI-X) bus, a serial advanced technology attachment (SATA) bus, a video electronics standards association local bus (VLB) bus, or the like, or any combination thereof. In some embodiments, the bus may include one or more buses. Although specific buses are described, the present disclosure may consider any suitable bus or interconnection.

FIG. 3 is a schematic diagram illustrating exemplary hardware and/or software components of an exemplary mobile device 300 on which the terminal 130 may be implemented according to some embodiments of the present disclosure. As illustrated in FIG. 3, the mobile device 300 may include a communication unit 310, a display 320, a graphics processing unit (GPU) 330, a central processing unit (CPU) 340, an I/O 350, a memory 360, and a storage 390. In some embodiments, any other suitable component, including but not limited to a system bus or a controller (not shown), may also be included in the mobile device 300. In some embodiments, a mobile operating system (OS) 370 (e.g., iOS™, Android™, Windows Phone™ etc.) and one or more applications (App(s)) 380 may be loaded into the memory 360 from the storage 390 in order to be executed by the CPU 340. The applications 380 may include a browser or any other suitable mobile apps for receiving and rendering information relating to image processing or other information from the processing device 140. User interactions with the information stream may be achieved via the I/O 350 and provided to the processing device 140 and/or other components of the imaging system 100 via the network 120. In some embodiments, a user may input parameters to the imaging system 100, via the mobile device 300.

In order to implement various modules, units, and their functions described above, a computer hardware platform may be used as hardware platforms of one or more elements (e.g., the processing device 140 and/or other components of the imaging system 100 described in FIG. 1). Since these hardware elements, operating systems, and program languages are common; it may be assumed that persons skilled in the art may be familiar with these techniques and they may be able to provide information needed in the image processing operations according to the techniques described in the present disclosure. A computer with the user interface may be used as a personal computer (PC), or other types of workstations or terminal devices. After being properly programmed, a computer with the user interface may be used as a server. It may be considered that those skilled in the art may also be familiar with such structures, programs, or general operations of this type of computing device.

FIG. 4A is a schematic diagram illustrating an exemplary imaging device 400 according to some embodiments of the present disclosure. The imaging device 400 may be an exemplary embodiment of the imaging device 110 as described in connection with FIG. 1. As shown in FIG. 4A, the imaging device 400 may include a supporting assembly 410, a detector assembly 420, a table 440, and a radiation emitting assembly 450. A subject may be placed on the table 440 and be transmitted along a first direction (denoted by a Z axis as shown in FIG. 4) to a detection region 430 of the imaging device 400 for imaging.

The supporting assembly 410 may support the detector assembly 420 and the radiation emitting assembly 450. For example, the detector assembly 420 and the radiation emitting assembly 450 may be both disposed on the supporting assembly 410. In some embodiments, the supporting assembly 410 may include a gantry. The gantry may include a housing that forms the detection region 430. In some embodiments, the gantry may further include a main frame, one or more support components, or the like, or any combination thereof.

In some embodiments, the radiation emitting assembly 450 may include a plurality of imaging sources configured to irradiate the subject simultaneously. For example, the radiation emitting assembly 450 may include two imaging sources, three imaging sources, four imaging sources, etc. A count (or number) of the plurality of imaging sources may be determined based on actual conditions. For example, the count (or number) of the plurality of imaging sources may be determined based on a required temporal resolution. The more the imaging sources, the higher the temporal resolution may be.

In some embodiments, two adjacent imaging sources among the plurality of imaging sources may form an included angle. The included angle may refer to an angle between connecting lines of the two adjacent imaging sources and a center of the detection region 430. For example, as shown in FIG. 4A, the radiation emitting assembly 450 may include a first imaging source 451 and a second imaging source 452, wherein an included angle between the first imaging source 451 and the second imaging source 452 may be 90 degrees. As another example, referring to FIG. 4B, FIG. 4B is a schematic diagram illustrating another exemplary radiation emitting assembly 460 according to some embodiments of the present disclosure. The radiation emitting assembly 460 may include a first imaging source 461, a second imaging source 462, and a third imaging source 463, wherein an included angle between two adjacent imaging sources among the first imaging source 461, the second imaging source 462, and the third imaging source 463 may be 60 degrees. As still another example, referring to FIG. 4C, FIG. 4C is a schematic diagram illustrating another exemplary radiation emitting assembly 470 according to some embodiments of the present disclosure. The radiation emitting assembly 470 may include a first imaging source 471, a second imaging source 472, and a plurality of third imaging sources 473-478, wherein an included angle between two adjacent imaging sources among the first imaging source 471, the second imaging source 472, and the plurality of third imaging sources 473-478 may be 45 degrees.

In some embodiments, the included angles between different pairs of adjacent imaging sources may be the same or different. In some embodiments, the included angle between adjacent imaging sources may be determined based on the count (or number) of the plurality of imaging sources. In some embodiments, a relative position between the plurality of imaging sources may be still during the rotation of the radiation emitting assembly 450. For example, the included angle of the first imaging source 451 and the second imaging source 452 may maintain 90 degrees during the scanning. As another example, referring to FIG. 4B, the included angle of two adjacent imaging sources among the first imaging source 461, the second imaging source 462, and the third imaging source 463 may maintain 60 degrees during the scanning.

Each of the plurality of imaging sources may be configured to emit radiation rays (e.g., X-ray beams, gamma-ray beams, etc.) having a plurality of energy levels for irradiating the subject in a scanning angle range. For example, the energy levels may include two or more of 10 kilovolts (kV), 20 kV, 30 kV, 40 kV, 50 kV, 60 kV, 70 kV, 80 kV, 90 kV, 100 kV, 120 kV, 140 kV, 160 kV, or the like, or any combination thereof.

The scanning angle range of an imaging source may refer to an angle range or a span of the angle range that the imaging source moves when the imaging source scans the subject in a scanning cycle. For example, if the imaging source moves from a first gantry angle to a second gantry angle during the scan, the scanning angle range of the imaging source may be from the first gantry angle to the second gantry angle. Merely by way of example, during the scan, the first imaging source 451 may rotate around the subject from 0 degrees to 90 degrees, and its scanning angle range may be 0 degrees to 90 degrees. In some embodiments, a span of the scanning angle range of each of the two imaging sources may be less than or equal to 90 degrees. Normally, an imaging source of a single-source imaging device needs to rotate around the subject for 180 degrees or more than 180 degrees to collect enough scanning data for reconstructing images with a desired quality, that is, a single scanning cycle (or referred to as a target scanning angle range of the subject) is 180 degrees or more than 180 degrees. If the imaging device includes two imaging sources, a span of a scanning angle range of each of the two imaging sources needs to be 90 degrees or more than 90 degrees. According to some embodiments of the present disclosure, the span of the scanning angle range of each of the two imaging sources may be less than 90 degrees, and images may be generated based on the scan data using a machine learning technique (e.g., at least one first image generation model). In other words, some embodiments of the present disclosure may achieve a desired reconstruction result with a relatively smaller amount of scan data by taking advantage of the machine learning technique, thereby improving the temporal resolution and reducing the radiation damage to the subject.

In some embodiments, the scanning angle range of the imaging source may be determined based on the count (or number) of the plurality of imaging sources and a total scanning angle range of a single scanning cycle. For example, if the count (or number) of the plurality of imaging sources is two and the total scanning angle range is 120 degrees, a scanning angle range of each of the two imaging sources may be 60 degrees. In some embodiments, the scanning angle ranges of different imaging sources may be the same or different.

In some embodiments, each of the plurality of imaging sources may include a ray emitter capable of emitting radiation rays having multiple energy levels, such as a switching imaging source (e.g., a kilovolt (kV) switching imaging source), a multi-beam imaging source (e.g., a twin-beam imaging source), etc. For example, the first imaging source 451 may be a fast kV switching imaging source including a cathode, an anode, and a switching arrangement (e.g., at least one high voltage switch). The anode may be configured to be selectively coupled to a plurality of voltage sources, and the switching arrangement may be configured to alternatingly couple the anode to the plurality of voltage sources. By controlling the switching arrangement, the kV switching imaging source (e.g., the fast kV switching imaging source) may sequentially emit sets of radiation rays having a plurality of energy levels. Each set of radiation rays having an energy level may correspond to one of the plurality of voltage sources. Merely by way of example, during the scan, the energy level of the imaging source 451 may alternate between 80 kV and 140 kV. In some embodiments, the plurality of energy levels may be switched in a certain frequency. For example, when the first imaging source 451 rotates 1 degree, a switching arrangement of the first imaging source 451 may be switched once. As another example, a duration of each energy level of the plurality of energy levels may be less than a duration threshold, such as, 0.1 milliseconds, 0.2 milliseconds, 0.5 milliseconds, etc.

In some embodiments, the switching patterns of the imaging source 451 and the imaging source 452 may be the same or different. In some embodiments, the switching patterns of the imaging source 451 and the imaging source 452 may be set such that the two imaging sources may switch their energy levels synchronously. In some embodiments, the imaging source 451 and the imaging source 452 may switch their energy levels rapidly, for example, every certain microsecond or scanning angle. In this way, the effect of the physiological motion of the subject may be obviated or reduced, and scan data of different energy levels may have a relatively higher spatial consistency.

In some embodiments, emitting durations of the radiation rays having the plurality of energy levels may be the same. For example, the scanning angle range of the first imaging source 451 may be from 0 degrees to 90 degrees, wherein energy levels corresponding to 0 to 1 degree, 2 to 3 degrees, 4 to 5 degrees, . . . and, 88 to 89 degrees are 80 kV; and energy levels corresponding to 1 to 2 degree, 3 to 4 degrees, 5 to 6 degrees, . . . and, 89 to 90 degrees are 140 kV. In some embodiments, emitting durations of the radiation rays having the plurality of energy levels may be different. For instance, switching patterns of the kV switching imaging source may be determined based on penetrating capacities of the plurality of energy levels. Normally, radiation rays having a high energy level may have higher penetrating capacities through a transmission medium (e.g., a human body) than radiation rays having a low energy level. Less scan data corresponding to the high energy level may need to be collected for reconstructing images having a desired image quality than the low energy level. Therefore, radiation rays having a low energy level may be assigned with a longer emission duration. For example, a kV switching imaging source (e.g., the fast kV switching imaging source) may switch between 80 kV and 140 kV, the switching pattern of the kV switching imaging source may be 80 kV, 80 kV, and 140 kV. In such cases, a ratio of the emitting duration corresponding to 140 kV and the emitting duration corresponding to 80 kV may be 1:2, etc. In this way, an image quality (e.g., a noise level) of an image reconstructed based on scan data corresponding to 140 kV may be the same as or similar to that of an image reconstructed based on scan data corresponding to 80 kV, which may improve an accuracy of a subsequent operation (e.g., a material separation operation).

A multi-beam imaging source may be able to emit mixed radiation rays including radiation rays having different energy levels. For example, the multi-beam imaging source may include a component (e.g., a split filter) configured to split the radiation rays into a high energy level and a low energy level before the radiation rays reaches the subject. For example, the split filter may include gold (Au) and tin (Sn). Therefore, the radiation rays having the plurality of energy levels may be used to irradiate the subject in the scanning angle range. Merely by way of example, the multi-beam imaging source may be a twin-beam imaging source that can emit mixed radiation rays for irradiating the subject in the scanning angle range. The mixed radiation rays may include a first portion having a first energy level (e.g., 80 kV) and a second portion having a second energy level (e.g., 140 kV).

The detector assembly 420 may be configured to detect radiation rays (e.g., X-ray photons, gamma photons, etc.) emitted from the radiation emitting assembly 450 after passing through the subject. In some embodiments, the detector assembly 420 may receive radiation rays (e.g., X-ray photons, gamma rays) and generate electrical signals. The radiation emitting assembly 450 and the detector assembly 420 may be used in conjunction to scan and image a certain portion of the subject for the diagnosis or treatment of various diseases.

The detector assembly 420 may include at least one detector. Each detector may include one or more detector rows arranged along a first direction (denoted by the Z axis as shown in FIG. 4A). Each detector row may include one or more detector modules. In some embodiments, a count (or number) of the at least one detector may be the same as the count (or number) of the plurality of imaging sources, and each detector may correspond to one of the imaging sources. Merely by way of example, as shown in FIG. 4A, the radiation emitting assembly 450 may include the first imaging source 451 and the second imaging source 452, and the detector assembly 420 may include a first detector 421 and a second detector 422. The first imaging source 451 and the second imaging source 452 may be configured to irradiate the subject simultaneously, wherein the first imaging source 451 is configured to emit first radiation rays having a plurality of first energy levels for irradiating the subject in a first scanning angle range, the second imaging source 452 is configured to emit second radiation rays having a plurality of second energy levels for irradiating the subject in a second scanning angle range, and the second scanning angle range is different from the first scanning angle range. In some embodiments, the plurality of first energy levels may be the same as the plurality of second energy levels. For example, the plurality of first energy levels may include 80 kV and 140 kV, and the plurality of second energy levels may also include 80 kV and 140 kV. In some embodiments, the plurality of first energy levels may be different from the plurality of second energy levels. For example, the plurality of first energy levels may include 80 kV and 140 kV, and the plurality of second energy levels may also include 90 kV and 150 kV. As another example, the plurality of first energy levels may include 80 kV and 140 kV, and the plurality of second energy levels may also include 80 kV and 150 kV. The first imaging source 451 may correspond to the first detector 421, and the second imaging source 452 may correspond to the second detector 422. That is, the first detector 421 may be configured to collect scan data of the subject by detecting the first radiation rays after passing through the subject, and the second detector 422 may be configured to collect scan data of the subject by detecting the second radiation rays after passing through the subject.

In some embodiments, the count (or number) of the at least one detector may be different from the count (or number) of the plurality of imaging sources. For example, the radiation emitting assembly 450 may include the first imaging source 451 and the second imaging source 452, and the detector assembly 420 may include a single detector. A first portion of the detector may correspond to the first imaging source 451, and a second portion of the detector may correspond to the second imaging source 452.

In some embodiments, the radiation emitting assembly 450 and the detector assembly 420 may rotate, for example, clockwise or counterclockwise about a rotation axis of the supporting assembly 410. For example, the supporting assembly 410 may rotate clockwise or counterclockwise about its rotation axis, and the radiation emitting assembly 450 and the detector assembly 420 may rotate together with the supporting assembly 410. As another example, the radiation emitting assembly 450 and the detector assembly 420 may be driven to rotate along a rail of the supporting assembly 410. In some embodiments, a relative position between the radiation emitting assembly 450 and the detector assembly 420 may be still during the rotation of the radiation emitting assembly 450 and the detector assembly 420. For example, a line connecting the first imaging source 451 and a center of the first detector 421 and a line connecting the second imaging source 452 and a center of the second detector 422 may pass through the center of the detection region 430 during the scanning, respectively. More descriptions regarding the rotation of the radiation emitting assembly 450 and the detector assembly 420 may be found elsewhere in the present disclosure (e.g., FIGS. 5A-5B and the descriptions thereof).

In some embodiments, the radiation emitting assembly 450 may be static during the scan. For example, a plurality of imaging sources of the radiation emitting assembly 450 may be arranged around the detection region 430, so that the plurality of imaging sources can scan the subject in the scanning cycle. Merely by way of example, referring to FIG. 4C, the first imaging source 471, the second imaging source 472, and the plurality of third imaging sources 473-478 may be arranged around the detection region 430, and the plurality of imaging sources (i.e., the first imaging source 471, the second imaging source 472, and the plurality of third imaging sources 473-478) may scan the subject in the scanning cycle. Therefore, the first imaging source 471, the second imaging source 472, and the plurality of third imaging sources 473-478 may be static during the scan. In some embodiments, each of the plurality of third imaging sources 473-478 may be configured to emit third radiation rays having one or more energy levels for irradiating the subject in a portion of a third scanning angle range, and the third scanning angle range may be different from the first scanning angle range and the second scanning angle range. For example, radiation rays emitted by the first imaging source 471, the third imaging source 473, the third imaging source 475, and the third imaging source 477 may have a first energy level (e.g., 140 eV), and radiation rays emitted by the second imaging source 472, the third imaging source 474, the third imaging source 476, and the third imaging source 478 may have a second energy level (e.g., 80 eV). The third scanning angle range, the first scanning angle range, and the second scanning angle range may form the scanning cycle. By arranging the plurality of imaging sources of the radiation emitting assembly 450, the scanning efficiency may be improved.

In some embodiments, each of the plurality of third imaging sources may be the same as the first imaging source or the second imaging source. For example, the plurality of third imaging sources, the first imaging source, and the second imaging source may be multi-beam imaging sources. As another example, the plurality of third imaging sources, the first imaging source, and the second imaging source may be single beam imaging sources. As another example, the plurality of third imaging sources, the first imaging source, and the second imaging source may be kV switching imaging sources. In some embodiments, each of the plurality of third imaging sources may be different from the first imaging source or the second imaging source. For example, the first imaging source and the second imaging source may be multi-beam imaging sources, and each of the plurality of third imaging sources may be a single beam imaging source.

By collecting the scan data of the subject using the plurality of imaging sources, the temporal resolution of the imaging device may be improved. In addition, each of the plurality of imaging sources may emit the radiation rays having the plurality of energy levels (e.g., the plurality of first energy levels, the plurality of second energy levels, etc.), that is, a muti-energy imaging technique may be combined with a multi-source imaging technique. Therefore, a plurality of images corresponding to the plurality of energy levels may be reconstructed based on the scan data, which may provide additional information beyond images reconstructed based on scan data that is obtained with radiation rays having a single energy level. Furthermore, since the scan data of different energy levels are collected substantially at the same time, spatial structures in the plurality of images of different energy levels may be consistent, which may improve the accuracy of image registration and subsequent material separation performed based on the image registration.

It should be noted that the imaging device 400 is provided for illustration purposes, and not intended to limit the scope of the present disclosure. For persons having ordinary skills in the art, multiple variations and modifications may be made under the teachings of the present disclosure. However, those variations and modifications do not depart from the scope of the present disclosure. For example, the plurality of imaging sources may be located in a different plane parallel to an XY plane. As another example, the counts of the radiation emitting assembly 450 and the detector assembly 420 may be modified. As still another example, the included angle between two adjacent imaging sources among the plurality of imaging sources may be altered according to actual conditions. For instance, the included angle between the first imaging source 451 and the second imaging source 452 may be 60 degrees for improving the temporal resolution.

FIG. 5A is a schematic diagram illustrating an exemplary scanning process of an imaging device 500 according to some embodiments of the present disclosure.

As shown in FIG. 5A, the imaging device 500 may include a first imaging source A and a second imaging source B. Both the first imaging source A and the second imaging source B may be kV switching imaging sources (e.g., fast kV switching imaging sources). “1” and “2” in FIG. 5A may represent a first energy level and a second energy level (e.g., 80 eV and 140 eV). The first imaging source A may rotate around a subject in a first scanning angle range (e.g., a range from 0 degrees to 90 degrees). When rotating in the first scanning angle range, the first imaging source A may alternate its energy level between the first energy level and the second energy level to irradiate the subject with first radiation rays having the first energy level and the second energy level. The second imaging source B may rotate around the subject in a second scanning angle range (e.g., a range from 90 degrees to 180 degrees). When rotating in the second scanning angle range, the second imaging source B may alternate its energy level between the first energy level and the second energy level to irradiate the subject with second radiation rays having the first energy level and the second energy level. For example, during the scan, the first imaging source A and the second imaging source B may rotate clockwise or counterclockwise about an axis of rotation of the imaging device 500, and irradiate the subject.

FIG. 5B is a schematic diagram illustrating an exemplary scanning process of an imaging device 550 according to some embodiments of the present disclosure.

As shown in FIG. 5B, the imaging device 500 may include a first imaging source C and a second imaging source D. Both the first imaging source C and the second imaging source D may be multi-beam imaging sources. For example, both the first imaging source C and the second imaging source D may be twin-beam imaging sources. The first imaging source C may emit first mixed radiation rays for irradiating the subject in a first scanning angle range (e.g., a range from 0 degrees to 90 degrees). The first mixed radiation rays may include a first portion having the first energy level and a second portion having the second energy level. The second imaging source D may emit second mixed radiation rays for irradiating the subject in a second scanning angle range (e.g., a range from 90 degrees to 180 degrees). The second mixed radiation rays may include a first portion having the first energy level and a second portion having the second energy level. For example, during the scan, the first imaging source C and the second imaging source D may rotate clockwise or counterclockwise about an axis of rotation of the imaging device 550, and irradiate the subject.

It should be noted that the imaging device 500 and the imaging device 550 are provided for illustration purposes, and not intended to limit the scope of the present disclosure. For persons having ordinary skills in the art, multiple variations and modifications may be made under the teachings of the present disclosure. However, those variations and modifications do not depart from the scope of the present disclosure. For example, the imaging device 500 and/or the imaging device 550 may include more than two imaging sources. As another example, a span of the scanning angle range (e.g., the first scanning angle range, the second scanning angle range, etc.) may be less than 90 degrees.

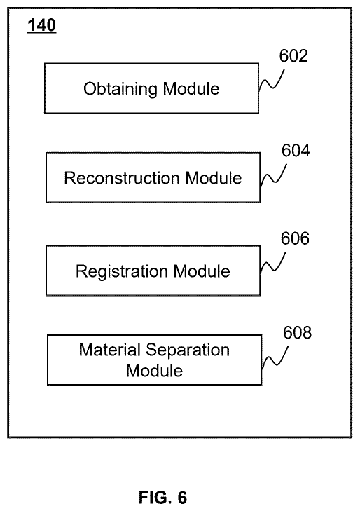

FIG. 6 is a block diagram illustrating an exemplary processing device 140 according to some embodiments of the present disclosure. In some embodiments, the processing device 140 may be in communication with a computer-readable storage medium (e.g., the storage device 150 illustrated in FIG. 1, or the storage device 220 illustrated in FIG. 2) and may execute instructions stored in the computer-readable storage medium. The processing device 140 may include an obtaining module 602, a reconstruction module 604, a registration module 606, and a material separation module 608.

The obtaining module 602 may be configured to obtain scan data of a subject. The scan data of the subject may include a plurality of sets of scan data each of which corresponds to one of a plurality of energy levels. More descriptions regarding the obtaining of the scan data of the subject may be found elsewhere in the present disclosure. See, e.g., operation 702 and relevant descriptions thereof.

The reconstruction module 604 may be configured to generate reconstruction images corresponding to the plurality of energy levels based on the plurality of sets of scan data. A reconstruction image of a certain energy level may be reconstructed based on a group of scan data corresponding to the energy level. The group of scan data may include a portion of the plurality of sets of scan data corresponding to the energy level. In some embodiments, the reconstruction module 604 may generate the reconstruction images using an image reconstruction technique (or algorithm). More descriptions regarding the generation of the reconstruction images may be found elsewhere in the present disclosure. See, e.g., operation 704 and relevant descriptions thereof.

The registration module 606 may be configured to obtain registered reconstruction images by performing a registration operation on the reconstruction images. The registration operation may refer to an operation of transforming different sets of scan data into one coordinate system. More descriptions regarding the obtaining of the registered reconstruction images may be found elsewhere in the present disclosure. See, e.g., operation 706 and relevant descriptions thereof.

The material separation module 608 may be configured to generate one or more material-specific images by performing a material separation on the registered reconstruction images. A material-specific image may refer to an image including information relating to a specific material of the subject. The material separation may refer to an operation that determines information of separate materials from the same group of data. More descriptions regarding the generation of the one or more material-specific images may be found elsewhere in the present disclosure. See, e.g., operation 708 and relevant descriptions thereof.

It should be noted that the above descriptions of the processing device 140 are provided for the purposes of illustration, and not intended to limit the scope of the present disclosure. For persons having ordinary skills in the art, various variations and modifications may be conducted under the guidance of the present disclosure. However, those variations and modifications do not depart from the scope of the present disclosure. In some embodiments, the processing device 140 may include one or more other modules. For example, the processing device 140 may include a storage module to store data generated by the modules in the processing device 140. In some embodiments, any two of the modules may be combined as a single module, and any one of the modules may be divided into two or more units.

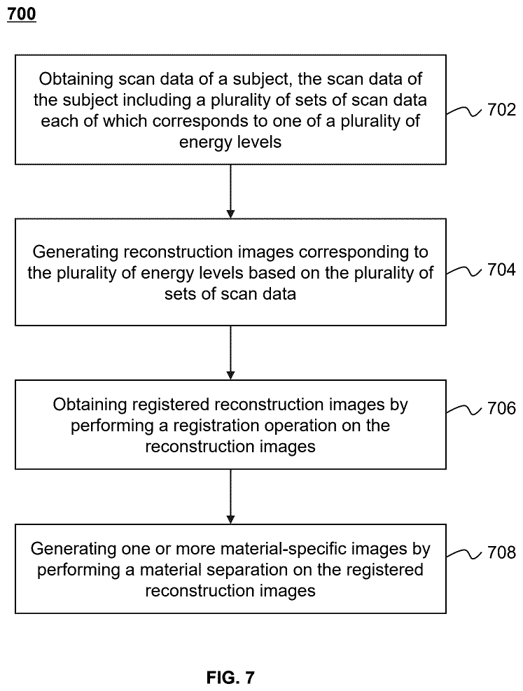

FIG. 7 is a flowchart illustrating an exemplary process for generating one or more material-specific images according to some embodiments of the present disclosure. Process 700 may be implemented in the imaging system 100 illustrated in FIG. 1. For example, the process 700 may be stored in the storage device 150, the storage device 220, and/or the storage 390 in the form of instructions (e.g., an application), and invoked and/or executed by the processing device 140 (e.g., the processing device 140 illustrated in FIG. 1, or one or more modules in the processing device 140 illustrated in FIG. 6). The operations of the illustrated process presented below are intended to be illustrative. In some embodiments, the process 700 may be accomplished with one or more additional operations not described, and/or without one or more of the operations discussed. Additionally, the order in which the operations of the process 700 as illustrated in FIG. 7 and described below is not intended to be limiting.

In 702, the processing device 140 (e.g., the obtaining module 602) may obtain scan data of a subject.

The scan data of the subject may include a plurality of sets of scan data each of which corresponds to one of a plurality of energy levels. The plurality of energy levels may include a plurality of first energy levels and a plurality of second energy levels. In some embodiments, the processing device 140 may obtain the scan data of the subject from an imaging device (e.g., the imaging device 400, the imaging device 460, the imaging device 500, the imaging device 550, etc.) or a storage device (e.g., the storage device 150, the storage device 220, the storage 390, or an external storage) that stores the scan data of the subject.

Merely by way of example, taking the imaging device 500 as an example, the first imaging source A may emit first radiation rays having 80 kV and 140 kV for irradiating the subject in a first scanning angle range from 0 degrees to 60 degrees, and the second imaging source B may emit second radiation rays having 80 kV and 140 kV for irradiating the subject in a second scanning angle range from 90 degrees to 150 degrees. Correspondingly, the scan data of the subject may include a first group of scan data of 80 kV and a second group of scan data of 140 kV. The first group of scan data may include a first portion of 80 kV collected in the first scanning angle range and a second portion of 80 kV collected in the second scanning angle range; and the second group of scan data includes a first portion of 140 kV collected in the first scanning angle range and a second portion of 140 kV collected in the second scanning angle range.

In 704, the processing device 140 (e.g., the reconstruction module 604) may generate reconstruction images corresponding to the plurality of energy levels based on the plurality of sets of scan data.

A reconstruction image of a certain energy level may be reconstructed based on a group of scan data corresponding to the energy level. The group of scan data may include a portion of the plurality of sets of scan data corresponding to the certain energy level. In some embodiments, the processing device 140 may generate the reconstruction images using an image reconstruction technique (or algorithm). Exemplary image reconstruction techniques may include a direct back projection technique, a filtered back projection technique, a convolutional back projection technique, a differential-Hilbert back projection technique, a gradient descent technique, an iterative reconstruction technique, or the like, or any combination thereof.

In some embodiments, the reconstruction images may further correspond to a target scanning angle range. The target scanning angle range may refer to an angle range having a desired span, such as 180 degrees or more than 180 degrees. In some embodiments, the target scanning angle range may be determined according to data requirements of the used image reconstruction algorithm or image quality requirements on the resulting image(s). Normally, for a single-source imaging device using parallel radiation beams or fan radiation beams, an imaging source needs to rotate around the subject for 180 degrees or more than 180 degrees to collect enough scanning data for image reconstruction (parallel beam projection data of 180 degrees or more than 180 degrees or fan-beam projection data having a fan angle of 180 degrees or more than 180 degrees). In such cases, a target scanning angle range may be set to have a span of 180 degrees or more than 180 degrees.

In some embodiments, the span of the total scanning angle range of the imaging sources of the multi-source imaging device may be less than the span of the target scanning angle range (e.g., 180 degrees). In such case, the processing device 140 may generate a plurality of preliminary reconstruction images corresponding to the plurality of energy levels based on the plurality of sets of scan data, and generate the reconstruction images by processing the plurality of preliminary reconstruction images.

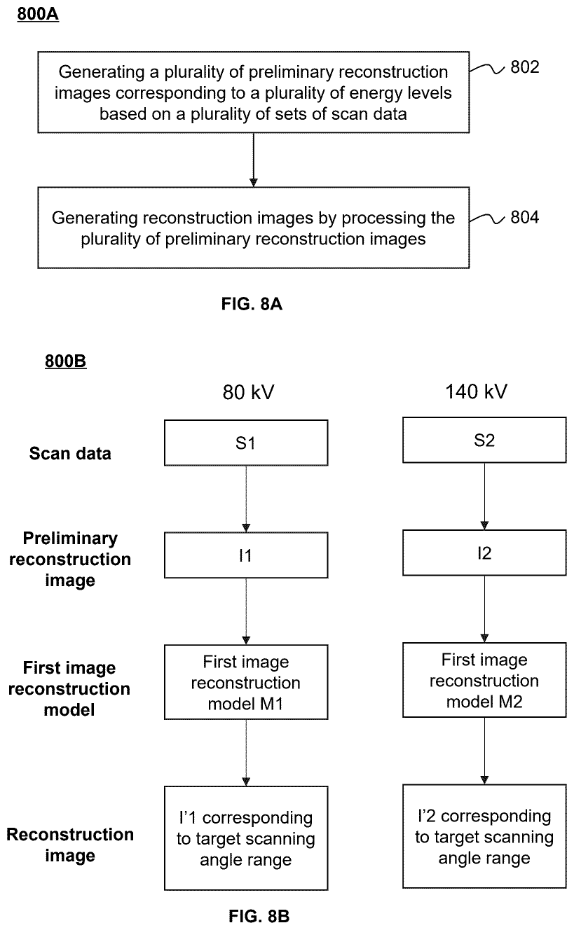

For example, for the imaging device 500 as aforementioned, the first scanning angle range of the first imaging source A may be from 0 degrees to 60 degrees, and the scanning angle range of the second imaging source B may be from 90 degrees to 150 degrees. Correspondingly, the span of the first group of scan data of 80 kV and the second group of scan data of 140 kV may be 120 degrees, which may be less than the target scanning angle range. The processing device 140 may generate a first preliminary reconstruction image corresponding to 80 kV and angle ranges from 0 to 60 degrees and from 90 to 120 degrees based on the first group of scan data, and a second preliminary reconstruction image corresponding to 140 kV and angle ranges from 0 to 60 degrees and from 90 to 150 degrees based on the second group of scan data. Further, the processing device 140 may process the first preliminary reconstruction image to generate a first reconstruction image corresponding to 80 kV and 180 degrees, and process the second preliminary reconstruction image using to generate a second reconstruction image corresponding to 140 kV and 180 degrees. As another example, an imaging device may include a first imaging source and a second imaging source, wherein the first imaging source may emit first radiation rays having 80 kV and 140 kV for irradiating a subject in a first scanning angle range from 0 degrees to 60 degrees, the second imaging source may emit second radiation rays having 80 kV and 140 kV for irradiating the subject in a second scanning angle range from 60 degrees to 120 degrees, and an included angle between the first imaging source and the second imaging source may be 60 degrees. Correspondingly, the first group of scan data of 80 kV and the second group of scan data of 140 kV may both correspond to a total scanning angle range from 0 to 120 degrees, the span of which may be less than the target scanning angle range. The processing device 140 may generate a first preliminary reconstruction image corresponding to 80 kV and an angle range from 0 to 120 degrees based on the first group of scan data, and a second preliminary reconstruction image corresponding to 140 kV and an angle range from 0 to 120 degrees based on the second group of scan data. Further, the processing device 140 may process the first preliminary reconstruction image to generate a first reconstruction image corresponding to 80 kV and 180 degrees, and process the second preliminary reconstruction image using to generate a second reconstruction image corresponding to 140 kV and 180 degrees. More descriptions regarding the generation of the reconstruction images may be found elsewhere in the present disclosure (e.g., FIGS. 8A-8B and the descriptions thereof).

In some embodiments, the processing device 140 may perform a pre-processing operation on the plurality of sets of scan data before generating the reconstruction images. For example, the processing device 140 may perform a weighting operation on the plurality of sets of scan data. Merely by way of example, the processing device 140 may generate a plurality of sets of weighted scan data by weighting the plurality of sets of scan data using a weighting model. The weighting model may indicate a corresponding relationship between weighting values and scanning angles. Exemplary weighting models may include a table, a diagram, a mathematic function, a curve, etc., that can represent the corresponding relationship between the weighting values and the scanning angles. For example, referring to FIG. 11, the weighting model may be a weighting curve including a first portion 1102 corresponding to the first scanning angle range of the first imaging source A and a second portion 1104 corresponding to the second scanning angle range of the second imaging source B, wherein an abscissa axis represents scanning angles of imaging sources (e.g., the first imaging source A and the second imaging source B), and an ordinate axis represents scanning angles corresponding to the scanning angles. As illustrated in FIG. 11, the weighting model may be an asymmetrical weighting curve. The weighting values may decrease slowly with the scanning angle in the first portion 1102, and then increase rapidly with the scanning angle in the second portion 1104. The processing device 140 may generate the reconstruction images corresponding to the plurality of energy levels based on the plurality of sets of weighted scan data.

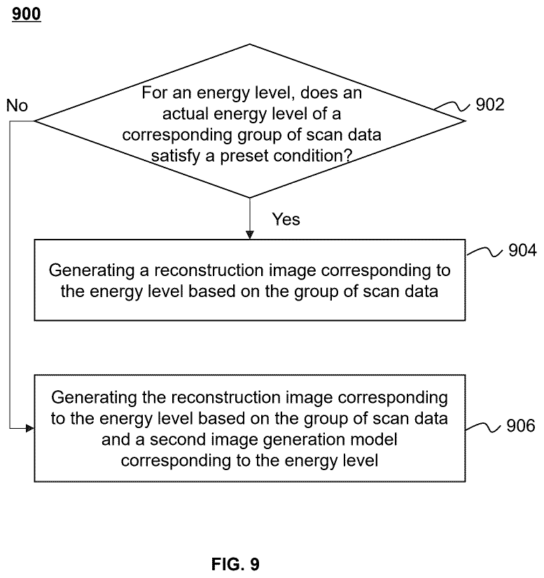

In some embodiments, the processing device 140 may perform data verification on the sets of scan data before generating the reconstruction images. For example, for each energy level, the processing device 140 may determine whether an actual energy level of the corresponding group of scan data satisfies a preset condition. If the actual energy level of the corresponding group of scan data satisfies the preset condition, the processing device 140 may generate the reconstruction image corresponding to the energy level based on the group of scan data of the energy level. If the actual energy level of the corresponding group of scan data doesn't satisfy the preset condition, the processing device 140 may generate the reconstruction image corresponding to the energy level based on the group of scan data and a second image generation model corresponding to the energy level. The second image generation model may be configured to generate the reconstruction image corresponding to the energy level based on the group of scan data. More descriptions regarding the generation of the reconstruction images may be found elsewhere in the present disclosure (e.g., FIG. 9 and the descriptions thereof).

In 706, the processing device 140 (e.g., the registration module 606) may obtain registered reconstruction images by performing a registration operation on the reconstruction images.

The registration operation may refer to an operation of transforming different sets of scan data into one coordinate system. The registration operation may be used to eliminate or reduce the spatial structure inconsistency between the reconstruction images, for example, caused by the physiological motion of the subject. In some embodiments, the processing device 140 may perform the registration operation on the reconstruction images. For example, the processing device 140 may register the reconstruction images using a registration algorithm. Exemplary registration algorithms may include a gray-scale information-based registration technique, a transform domain-based registration technique, a feature-based registration technique, an intensity-based registration algorithm, a machine learning model, or the like, or any combination thereof.

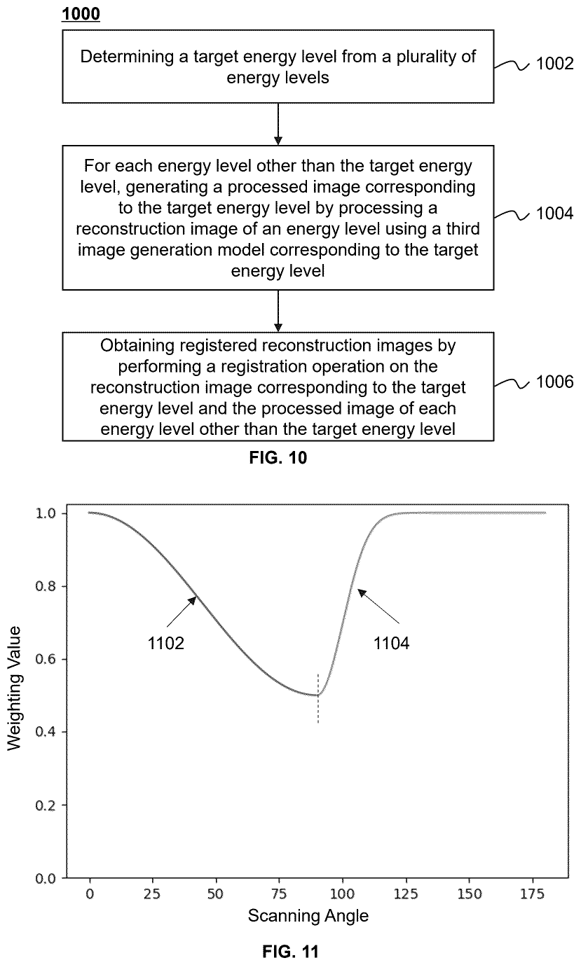

In some embodiments, before the registration operation, the processing device 140 may process the reconstruction images. For example, the processing device 140 may transform the reconstruction images corresponding to the plurality of energy levels into processed images corresponding to a same energy level. More descriptions regarding the obtaining of the registered reconstruction images may be found elsewhere in the present disclosure (e.g., FIG. 10 and the descriptions thereof).

In 708, the processing device 140 (e.g., the material separation module 608) may generate one or more material-specific images by performing a material separation on the registered reconstruction images.

A material-specific image may refer to an image including information relating to a specific material of the subject. The material-specific image(s) may provide qualitative and quantitative information about tissue composition and contrast media distribution of the subject. Exemplary material-specific images may include an iodine image, a water image, a calcium image, or the like, or any combination thereof. For example, in an iodine image, signals of the iodine may be strong, while signals of the water may be invisible. As another example, in a water image, signals of the water may be strong, while signals of the iodine may be invisible. The material separation may refer to an operation that determines information of

separate materials from the same set of data. For example, each voxel (or pixel) of the registered reconstruction images may be assumed to include two materials (e.g., water and iodine) in different proportions, wherein the proportions of the two materials within each voxel may be determined based on attenuation coefficients of the two materials at different energy levels and the registered reconstruction images. Therefore, the processing device 140 may generate an iodine image and a water image based on the proportions of the two materials within each voxel. In some embodiments, the material separation may be performed based on a two-material decomposition algorithm, a three-material decomposition algorithm, a multi-material decomposition (MMD) algorithm, etc.

In some embodiments, the processing device 140 may further process the one or more material-specific images. For example, the processing device 140 may perform a quantitative analysis on the one or more material-specific images for diagnosis or other purposes.

Some embodiments of the present disclosure, the reconstruction images corresponding to the plurality of energy levels may be obtained, and the one or more material-specific images may be further generated based on the reconstruction images. Therefore, additional information (e.g., the quantitative analysis of the one or more materials) may be provided, which may improve the accuracy of lesion detection, thereby improving the accuracy of diagnosis.

It should be noted that the description of the process 700 is provided for the purposes of illustration, and not intended to limit the scope of the present disclosure. For persons having ordinary skills in the art, various variations and modifications may be conducted under the teaching of the present disclosure. For example, operation 706 may be removed. That is, the reconstruction images may be performed the material separation without the registration operation. As another example, an additional operation for displaying the one or more material-specific images may be added after operation 708. However, those variations and modifications may not depart from the protection of the present disclosure.

FIG. 8A is a flowchart illustrating an exemplary process 800A for generating reconstruction images of a subject according to some embodiments of the present disclosure. In some embodiments, the process 800A may be performed to achieve at least part of operation 704 as described in connection with FIG. 7.

In 802, the processing device 140 (e.g., the reconstruction module 604) may generate a plurality of preliminary reconstruction images corresponding to a plurality of energy levels based on a plurality of sets of scan data corresponding to the energy levels.

As aforementioned, an imaging device (e.g., the imaging device 400, the imaging device 460, the imaging device 500, the imaging device 550, etc.) disclosed in the present disclosure may be used to collect the sets of scan data corresponding to different energy levels. Each preliminary reconstruction image may be a reconstruction image corresponding to an actual scanning angle range of the scan of the subject. In some embodiments, a span of the actual scanning angle range may be less than a span of the target scanning angle range. For example, the imaging device may include a first imaging source and a second imaging source, wherein the first imaging source has a first scanning angle range from 0 degrees to 60 degrees, and the second imaging source has a second scanning angle range from 60 degrees to 120 degrees. The span of the actual scanning angle range of the scan may be 120 degrees while the span of the target scanning angle range may be 180 degrees.

For each energy level, the processing device 140 may generate a corresponding preliminary reconstruction image based on a group of scan data corresponding to the energy level among the plurality of sets of scan data. In some embodiments, the processing device 140 may generate the plurality of preliminary reconstruction images using an image reconstruction technique as described elsewhere in this disclosure (e.g., FIG. 7 and the relevant descriptions).

In 804, the processing device 140 (e.g., the reconstruction module 604) may generate reconstruction images by processing the plurality of preliminary reconstruction images.

Each of the reconstruction images may correspond to the target scanning angle range. A reconstruction image corresponding to the target scanning angle range may be deemed as having the same or substantially the same image quality as an image reconstructed based on scan data that is collected with the target scanning angle range.

In some embodiments, the processing device 140 may generate the reconstruction images by processing the plurality of preliminary reconstruction images using at least one first image generation model corresponding to the target scanning angle range. A first image generation model corresponding to the target scanning angle range may be configured to transform an image corresponding to a preliminary scanning angle range into a reconstruction image corresponding to the target scanning angle range, wherein the preliminary scanning angle range is different from the target scanning angle range. In some embodiments, a universal first image generation model applicable to any energy level may be used. That is, different energy levels may correspond to the same first image generation model. The processing device 140 may generate the reconstruction images by processing the plurality of preliminary reconstruction images using the same universal first image reconstruction model. For example, the processing device 140 may input the plurality of preliminary reconstruction images into the universal first image reconstruction model, respectively, and the universal first image reconstruction model may output the reconstruction images.

In some embodiments, the at least one first image reconstruction model may include a plurality of first image reconstruction models each of which corresponds to one of the plurality of energy levels. A first image reconstruction model of a certain energy level may be configured to generate a reconstruction image corresponding to the energy level based on a preliminary reconstruction image corresponding to the energy level. For example, if the sets of scan data may include two groups corresponding to 80 kV and 140 kV, the at least one first image reconstruction model may include a first image reconstruction model corresponding to 80 kV and a first image reconstruction model corresponding to 140 kV.

In some embodiments, for each of the plurality of energy levels, the processing device 140 may select the first image reconstruction model of the energy level from the plurality of first image reconstruction models. For example, for 80 kV, the processing device 140 may select the first image reconstruction model corresponding to 80 kV; and for 140 kV, the processing device 140 may select the first image reconstruction model corresponding to 140 kV.

In some embodiments, for each of the plurality of energy levels, the processing device 140 may generate the reconstruction image corresponding to the energy level by processing the preliminary reconstruction image of the energy level using the selected first image reconstruction model. For example, the processing device 140 may input the preliminary reconstruction image of 80 kV into the selected first image reconstruction model corresponding to 80 kV, and the selected first image reconstruction model may output the reconstruction image of 80 kV. Since images corresponding to different energy levels may have different features, using the plurality of first image reconstruction models corresponding to the plurality of energy levels may improve the accuracy of the generated reconstruction images. In addition, the complexity of each first image reconstruction model may be reduced, which, in turn, may reduce the training difficulty and improve the accuracy of the plurality of first image reconstruction models.

In some embodiments, the at least one first image generation model may include a machine learning model, such as a convolutional neural network (CNN), a U-net model, a V-net model, a multi-layer perception machine, a support vector machine (SVM), a Bayes model, an Adaboost model, a logic regression model, a generative adversarial net (GAN), etc.

In some embodiments, a first image generation model may be generated according to a first training process. The first training process may include obtaining a plurality of first training samples. Each of the plurality of first training samples may include a first sample reconstruction image corresponding to a preliminary scanning angle range and a second sample reconstruction image corresponding to the target scanning angle range. For example, the first sample reconstruction image may be obtained based on a sample group of scan data collected under the preliminary scanning angle range, and the second sample reconstruction image may be obtained based on a sample group of scan data collected under the target scanning angle range. In some embodiments, the preliminary scanning angle range may be the same as the actual scanning angle range of the sets of scan data. In some embodiments, the span of the preliminary scanning angle range may be equal to a sum of the spans of the scanning angle range of the imaging sources of the imaging device.

In some embodiments, the first training process may further include generating the first image reconstruction model by training an initial model using the plurality of first training samples. The initial model may refer to a model whose parameter values are initialized. For example, first sample reconstruction images may be input into the initial model, and predicted reconstruction images may be outputted by the initial model. By adjusting the parameters of the initial model to reduce a difference between the predicted reconstruction images and the second sample reconstruction images, the initial model (i.e., parameter values of the initial model) may be iteratively updated, and finally, the first image reconstruction model may be obtained.

In some embodiments, the processing device 140 may generate the reconstruction images by processing the plurality of preliminary reconstruction images using an iterative reconstruction technique. For example, the processing device 140 may process each of the plurality of preliminary reconstruction images according to an iterative reconstruction equation (1):

$$E ( U ) = {‖ AU - Y ‖}_{{W}_{s}}^{2} + β \times R ( U ) , \qquad ( 1 )$$

where U denotes the reconstruction image to be generated, A denotes a projection matrix, Y denotes a group of scan data corresponding to the preliminary reconstruction image, Ws denotes weighting values corresponding to sets of scan data in the group of scan data (e.g., weighting values corresponding to scanning angles in the target scanning angle range), R(U) denotes a regularization term for the reconstruction image, and β denotes a coefficient regulating an intensity of the regularization term.

For instance, the processing device 140 may generate the reconstruction images by iteratively minimizing a value (i.e., E(U)) of the iterative reconstruction equation (1).

According to some embodiments of the present disclosure, the sets of scan data of the subject may be collected by an imaging device having a plurality of imaging sources, and the sets of scan data may correspond to an actual scanning angle range smaller than a target scanning angle range. Preliminary reconstruction images may be generated based on the sets of scan data and transformed into reconstruction images of the target scanning angle range using one or more first image generation models. By using the first image reconstruction model(s), reconstruction images having a desired image quality may be generated based on a relatively smaller scanning angle range. In this way, the scanning efficiency may be improved and the radiation dose delivered to the subject during the scan may be reduced.

It should be noted that the description of the process 800A is provided for the purposes of illustration, and not intended to limit the scope of the present disclosure. For persons having ordinary skills in the art, various variations and modifications may be conducted under the teaching of the present disclosure. However, those variations and modifications may not depart from the protection of the present disclosure.

FIG. 8B is a schematic diagram illustrating an exemplary process 800B for generating reconstruction images of a subject according to some embodiments of the present disclosure. The process 800B may be an exemplary embodiment of the process 800A as described in connection with FIG. 8.

As shown in FIG. 8B, scan data of a subject may include a group of scan data S1 corresponding to 80 kV (as an exemplary first energy level) and a group of scan data S2 corresponding to 140 kV (as an exemplary second energy level). Merely by way of example, a first imaging source may switch between 80 kV and 140 kV to scan the subject from 0 degrees to 60 degrees, and a second imaging source may switch between 80 kV and 140 kV to scan the subject from 60 degrees to 120 degrees. The scan data S1 and the scan data S2 may both correspond to an actual scanning angle range of 120 degrees.

For 80 kV, a preliminary reconstruction image I1 having the energy level of 80 kV and the scanning angle range of 120 degrees may be generated based on the scan data S1, a first image reconstruction model M1 corresponding to 80 kV may be selected, and a reconstruction image I′1 may be generated by processing the preliminary reconstruction image I1 using the first image reconstruction model M1. Similarly, for 140 kV, a preliminary reconstruction image I2 having the energy level of 140 kV and the scanning angle range of 120 degrees may be generated based on the scan data S2, a first image reconstruction model M2 corresponding to 140 kV may be selected, and a reconstruction image I′2 may be generated by processing the preliminary reconstruction image I2 using the first image reconstruction model M2.

It should be noted that the process 800B is provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. For example, a first image reconstruction model may correspond to the plurality of energy levels. That is, a single first image reconstruction model may be configured to process both the preliminary reconstruction image I1 and the preliminary reconstruction image I2.

FIG. 9 is a flowchart illustrating an exemplary process for generating a reconstruction image corresponding to an energy level according to some embodiments of the present disclosure. In some embodiments, for each of a plurality of energy levels, the process 900 may be performed to achieve operation 704 as described in connection with FIG. 7.

In 902, for an energy level, the processing device 140 (e.g., the reconstruction module 604) may determine whether an actual energy level of a corresponding group of scan data satisfies a preset condition.

During a scan of a subject, an imaging source of an imaging device is expected to switch its energy level exactly according to a switching pattern, such as switches between 80 kV and 140 kV every 2 microseconds. However, in some occasions, the actual energy level of the imaging source may be different from the group energy level (or referred to as a desired energy level). For example, the energy level may be 80 kV, while the actual energy level may be 78 kV. A difference between the actual energy level and the energy level may reduce the quality of the scan data, which reduces the image quality of resulting images and the accuracy of diagnosis. Therefore, before processing (e.g., reconstructing) the corresponding group of scan data, the processing device 140 may determine whether the actual energy level of the corresponding group of scan data satisfies the preset condition.

Taking 80 kV as an example, the preset condition may include that a difference between the actual energy level of the corresponding group of scan data and 80 kV is less than a difference threshold, a ratio of the actual energy level of the corresponding group of scan data and 80 kV is larger than a ratio threshold, or the like, or any combination thereof. In some embodiments, the difference threshold may be a value, such as, 0.01 kV, 0.05 kV, 0.1 kV, 0.2 kV, 0.5 kV, 1 kV, 2 kV, or the like. As another example, the difference threshold may be a percentage, such as, 80%, 85%, 90%, 95%, 99%, 99.5%, 99.9%, or the like. In some embodiments, the difference threshold and/or the ratio threshold may be determined based on the system default setting, or set manually by the user (e.g., a technician, a doctor, a physicist, etc.), or determined according to an actual need.

If the actual energy level of the corresponding group of scan data satisfies the preset condition, the process 900 may proceed to operation 904. In 904, the processing device 140 (e.g., the reconstruction module 604) may generate a reconstruction image corresponding to the energy level based on the group of scan data of the energy level.

If the actual energy level of the corresponding group of scan data doesn't satisfy the preset condition, the process 900 may proceed to operation 906. In 906, the processing device 140 (e.g., the reconstruction module 604) may generate the reconstruction image corresponding to the energy level based on the group of scan data and a second image generation model corresponding to the energy level. For example, if the energy level is 80 kV, the processing device 140 may generate the reconstruction image corresponding to 80 kV based on the group of scan data and the second image generation model corresponding to 80 kV.

In some embodiments, the processing device 140 may generate a preliminary reconstruction image based on the group of scan data of the energy level. For example, the processing device 140 may generate the preliminary reconstruction image using an image reconstruction technique as described elsewhere in this disclosure (e.g., FIG. 7 and the relevant descriptions).

In some embodiments, the processing device 140 may further generate the reconstruction image corresponding to the energy level by processing the preliminary reconstruction image using the second image generation model corresponding to the energy level. The second image reconstruction model corresponding to the energy level may be configured to generate the reconstruction image corresponding to the energy level based on the preliminary reconstruction image corresponding to another energy level. For example, if the energy level is 80 kV, the processing device 140 may input the preliminary reconstruction image corresponding to the actual energy level (e.g., 78 kV) into the second image generation model corresponding to 80 kV, and the second image generation model corresponding to 80 kV may output the reconstruction image corresponding to 80 kV.

In some embodiments, the second image generation model corresponding to the energy level may include a machine learning-based model, such as a convolutional neural network (CNN), a U-net model, a V-net model, a multi-layer perception machine, a support vector machine (SVM), a Bayes model, an Adaboost model, a logic regression model, a generative adversarial net (GAN), etc.

In some embodiments, the second image generation model corresponding to the energy level may be generated according to a second training process. The second training process may include obtaining a plurality of second training samples. Each of the plurality of second training samples may include a third sample reconstruction image corresponding to a preliminary energy level and a fourth sample reconstruction image corresponding to the energy level. For example, if the energy level is 80 kV, the third sample reconstruction image may be obtained based on a sample group of scan data corresponding to the preliminary energy level (e.g., any energy levels other than 80 kV), and the fourth sample reconstruction image may be obtained based on a sample group of scan data corresponding to 80 kV.

In some embodiments, the second training process may further include generating the second image reconstruction model by training a second initial model using the plurality of second training samples. The second initial model may refer to a model whose parameter values are initialized. For example, third sample reconstruction images may be input into the second initial model, and predicted reconstruction images may be outputted by the second initial model. By adjusting parameters of the second initial model to reduce a difference between the predicted reconstruction images and the fourth sample reconstruction images, the second initial model (i.e., parameter values of the second initial model) may be iteratively updated, and finally, the second image reconstruction model may be obtained.

According to some embodiments of the present disclosure, for an energy level, a data verification operation may be performed to determine whether an actual energy level of a corresponding group of scan data satisfies a preset condition. If the actual energy level doesn't satisfy the preset condition, a second image reconstruction model of the energy level may be used to generate the reconstruction image of the energy level. In this way, the effect of a deviation of the actual energy level from the energy level may be reduced or eliminated, thereby improving the data accuracy and the imaging quality.

It should be noted that the descriptions of the process 900 is provided for the purposes of illustration, and not intended to limit the scope of the present disclosure. For persons having ordinary skills in the art, various variations and modifications may be conducted under the teaching of the present disclosure. However, those variations and modifications may not depart from the protection of the present disclosure.

FIG. 10 is a flowchart illustrating an exemplary process for obtaining registered reconstruction images according to some embodiments of the present disclosure. In some embodiments, the process 1000 may be performed to achieve at least part of operation 706 as described in connection with FIG. 7.

As described in connection with operation 706, reconstruction images of different energy levels need to be registered to reduce or eliminate the spatial structure inconsistency between the reconstruction images caused by the physiological motion of the subject. Except for the physiological, the difference between the energy levels of the reconstruction images may also cause inconsistency between the reconstruction images. The inconsistency caused by the energy level difference may affect the registration accuracy. In order to improve the registration accuracy, the reconstruction images corresponding to different energy levels may be transformed into images corresponding to a same energy level before the registration by performing the process 1000.

In 1002, the processing device 140 (e.g., the registration module 606) may determine a target energy level from a plurality of energy levels.

The target energy level may be any energy level selected from the energy level.

In some embodiments, the processing device 140 may determine the target energy level according to actual conditions. For example, if the image quality of a reconstructed image corresponding to 140 kV is larger than the image quality of a reconstructed image corresponding to 80 kV, 140 kV may be determined as the target energy level.

In 1004, for each energy level other than the target energy level, the processing device 140 (e.g., the registration module 606) may generate a processed image corresponding to the target energy level by processing the reconstruction image of the energy level using a third image generation model corresponding to the target energy level.

The third image reconstruction model corresponding to the target energy level may be configured to generate a processed image corresponding to the target energy level based on a reconstruction image corresponding to the energy level other than the target energy level. For example, if the plurality of energy levels include 80 kV and 140 kV, and the target energy level is 140 kV, the processing device 140 may input a reconstruction image corresponding to 80 kV into a third image generation model corresponding to 140 kV, and the third image generation model corresponding to 140 kV may output a processed image corresponding to 140 kV.

In some embodiments, the third image generation model corresponding to the target energy level may include a machine learning-based model, such as a convolutional neural network (CNN), a U-net model, a V-net model, a multi-layer perception machine, a support vector machine (SVM), a Bayes model, an Adaboost model, a logic regression model, a generative adversarial net (GAN), etc.

In some embodiments, the third image generation model corresponding to the target energy level may be generated according to a third training process. The third training process may be similar to the second training process as described in FIG. 9.

In 1006, for each energy level other than the target energy level, the processing device 140 (e.g., the registration module 606) may obtain the registered reconstruction images by performing the registration operation on the reconstruction image corresponding to the target energy level and the processed image of each energy level other than the target energy level.

For example, if the plurality of energy levels include 80 kV and 140 kV, and the target energy level is 140 kV, the reconstruction image corresponding to 80 kV may be transformed into a processed image corresponding to 140 kV. The processing device 140 may obtain the registered reconstruction images by performing the registration operation on the reconstruction image corresponding to 140 kV and the processed image corresponding to 140 kV using a registration algorithm as described elsewhere in this disclosure (e.g., FIG. 7 and the relevant descriptions).

Some embodiments of the present disclosure, the reconstruction images corresponding to the plurality of energy levels may be transformed into the processed images corresponding to a same energy level. Therefore, an inconsistency caused by the energy level difference may be eliminated, which improves an accuracy of the registration.

It should be noted that the descriptions of the process 1000 are provided for the purposes of illustration, and not intended to limit the scope of the present disclosure. For persons having ordinary skills in the art, various variations and modifications may be conducted under the teaching of the present disclosure. However, those variations and modifications may not depart from the protection of the present disclosure.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended for those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various portions of this disclosure are not necessarily all referring to the same embodiment. Furthermore, the particular features, structures, or characteristics may be combined as suitable in one or more embodiments of the present disclosure.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed subject matter requires more features than are expressly recited in each claim. Rather, inventive embodiments lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

1. An imaging system, comprising:
a first imaging source and a second imaging source configured to irradiate a subject simultaneously, wherein the first imaging source is configured to emit first radiation rays having a plurality of first energy levels for irradiating the subject in a first scanning angle range, the second imaging source is configured to emit second radiation rays having a plurality of second energy levels for irradiating the subject in a second scanning angle range, and the second scanning angle range is different from the first scanning angle range; and
at least one detector configured to collect scan data of the subject by detecting the first radiation rays and the second radiation rays after passing through the subject.

2. The system of claim 1, wherein the first imaging source and the second imaging source are kV switching imaging sources.

3. The system of claim 2, wherein switching patterns of the first imaging source are determined based on penetrating capacities of the plurality of first energy levels, and switching patterns of the second imaging source are determined based on penetrating capacities of the plurality of second energy levels.

4. The system of claim 1, wherein the first imaging source and the second imaging source are multi-beam imaging sources.

5. The system of claim 1, further comprising:
a plurality of third imaging sources configured to irradiate the subject with the first imaging source and the second imaging source, simultaneously, wherein each of the plurality of third imaging sources is configured to emit third radiation rays having one or more energy levels for irradiating the subject in a portion of a third scanning angle range, and the third scanning angle range is different from the first scanning angle range and the second scanning angle range; and
the at least one detector further configured to collect the scan data of the subject by detecting the first radiation rays, the second radiation rays, and the third radiation rays after passing through the subject.

6. The system of claim 1, wherein the scan data of the subject includes a plurality of sets of scan data each of which corresponds to one of a plurality of energy levels, the plurality of energy levels including the plurality of first energy levels and the plurality of second energy levels and
the imaging system further includes a processing device configured to:
generate reconstruction images corresponding to the plurality of energy levels based on the plurality of sets of scan data.

7. The system of claim 6, wherein to generate reconstruction images corresponding to the plurality of energy levels based on the plurality of sets of scan data, the processing device is configured to:
generate a plurality of sets of weighted scan data by weighting the plurality of sets of scan data using a weighting model, the weighting model indicating a corresponding relationship between weighting values and scanning angles; and
generate the reconstruction images corresponding to the plurality of energy levels based on the plurality of sets of weighted scan data.

8. The system of claim 6, wherein the reconstruction images further correspond to a target scanning angle range, and to generate reconstruction images corresponding to the plurality of energy levels based on the plurality of sets of scan data, the processing device is configured to:
generate a plurality of preliminary reconstruction images corresponding to the plurality of energy levels based on the plurality of sets of scan data; and
generate the reconstruction images by processing the plurality of preliminary reconstruction images using at least one first image generation model corresponding to the target scanning angle range.

9. The system of claim 8, wherein the at least one first image reconstruction model includes a plurality of first image reconstruction models each of which corresponds to one of the plurality of energy levels, and
to generate the reconstruction images by processing the plurality of preliminary reconstruction images, the processing device is configured to:
for each of the plurality of energy levels,
select the first image reconstruction model of the energy level from the plurality of first image reconstruction models; and
generate the reconstruction image corresponding to the energy level by processing the preliminary reconstruction image of the energy level using the selected first image reconstruction model.

10. The system of claim 6, wherein to generate reconstruction images corresponding to the plurality of energy levels based on the plurality of sets of scan data, the processing device is configured to:
for each of the plurality of energy levels,
determine whether an actual energy level of a corresponding group of scan data satisfies a preset condition, the group of scan data including a portion of the plurality of sets of scan data corresponding to the energy level;
in response to determining that the energy level of the corresponding group of scan data satisfies the preset condition, generate the reconstruction image corresponding to the energy level based on the group of scan data of the energy level; or
in response to that the energy level of the corresponding group of scan data doesn't satisfy the preset condition, generate the reconstruction image corresponding to the energy level based on the group of scan data and a second image generation model corresponding to the energy level.

11. The system of claim 6, wherein the processing device is further configured to:
obtain registered reconstruction images by performing a registration operation on the reconstruction images; and
generate one or more material-specific images by performing a material separation on the registered reconstruction images.

12. The system of claim 11, wherein to obtain registered reconstruction images by performing a registration operation on the reconstruction images, the processing device is further configured to:
determine a target energy level from the plurality of energy levels;
for each energy level other than the target energy level, generate a processed image corresponding to the target energy level by processing the reconstruction image of the energy level; and
obtain the registered reconstruction images by performing the registration operation on the reconstruction image corresponding to the target energy level and the processed image of each energy level other than the target energy level.

13. The system of claim 12, wherein to process the reconstruction image of the energy level, the processing device is further configured to:
process the reconstruction image of the energy level using a third image generation model corresponding to the target energy level.

14. An imaging method, implemented on a computing device having at least one processor and at least one storage device, the method comprising:
directing a first imaging source and a second imaging source to irradiate a subject simultaneously, wherein the first imaging source is configured to emit first radiation rays having a plurality of first energy levels for irradiating a subject in a first scanning angle range, the second imaging source is configured to emit second radiation rays having a plurality of second energy levels for irradiating the subject in a second scanning angle range, and the second scanning angle range is different from the first scanning angle range; and
directing at least one detector to collect scan data of the subject by detecting the first radiation rays and the second radiation rays after passing through the subject.

15. The method of claim 14, wherein the first imaging source and the second imaging source are kV switching imaging sources.

16. The method of claim 14 or claim 15, further comprising:
directing a plurality of third imaging sources to irradiate the subject with the first imaging source and the second imaging source, simultaneously, wherein each of the plurality of third imaging sources is configured to emit third radiation rays having one or more energy levels for irradiating the subject in a portion of a third scanning angle range, and the third scanning angle range is different from the first scanning angle range and the second scanning angle range; and
directing the at least one detector to collect the scan data of the subject by detecting the first radiation rays, the second radiation rays, and the third radiation rays after passing through the subject.

17. The method of claim 14, wherein the scan data of the subject includes a plurality of sets of scan data each of which corresponds to one of the plurality of energy levels, and the method further includes:
generating reconstruction images corresponding to the plurality of energy levels based on the plurality of sets of scan data.

18. The method of claim 17, wherein the generating reconstruction images corresponding to the plurality of energy levels based on the plurality of sets of scan data includes:
generating a plurality of sets of weighted scan data by weighting the plurality of sets of scan data using a weighting model, the weighting model indicating a corresponding relationship between weighting values and scanning angles; and
generating the reconstruction images corresponding to the plurality of energy levels based on the plurality of sets of weighted scan data.

19. The method of claim 17, further comprising:
obtaining registered reconstruction images by performing a registration operation on the reconstruction images; and
generating one or more material-specific images by performing a material separation on the registered reconstruction images.

20. A non-transitory computer readable medium, comprising executable instructions that, when executed by at least one processor, direct the at least one processor to perform a method, the method comprising:
directing a first imaging source and a second imaging source to irradiate a subject simultaneously, wherein the first imaging source is configured to emit first radiation rays having a plurality of first energy levels for irradiating a subject in a first scanning angle range, the second imaging source is configured to emit second radiation rays having a plurality of second energy levels for irradiating the subject in a second scanning angle range, and the second scanning angle range is different from the first scanning angle range; and
directing at least one detector to collect scan data of the subject by detecting the first radiation rays and the second radiation rays after passing through the subject.
